(19) United States
(12) Patent Application Publication
(10) Pub. No.: US 2025/0160707 A1
(43) Pub. Date: May 22, 2025

(54) Device for Implementing a Non-Invasive Method for Determining the Homeostatic Emotional State of an Animal

(71) Applicants: ECOLE D'INGENIEURS DE PURPAN, Toulouse (FR); Institut national de recherche pour l'agriculture, l'alimentation et l'environnement, Paris (FR); ECOLE NATIONALE VETERINAIRE DE TOULOUSE, Toulouse (FR); UNIVERSITE TOULOUSE III - PAUL SABATIER, Toulouse (FR)

(72) Inventors: Hélène Eutamene, Balma (FR); Pauline Belloir, Pechabou (FR); Cécile Levasseur, Frouzins (FR); Djamila Lekhal, Toulouse (FR); Harold Clenet, Beauzelle (FR)

(21) Appl. No.: 18/707,146

(22) PCT Filed: Nov. 2, 2022
(86) PCT No.: PCT/FR2022/052065

§ 371 Date: May 2, 2024

(30) Foreign Application Priority Data
Nov. 4, 2021 (FR) FR2111719

(51) Int. Cl. A61B 5/16; A61B 5/00

(52) U.S. Cl. CPC A61B 5/16; A61B 5/0075; A61B 5/7203; A61B 5/7246; A61B 2503/40

(57) ABSTRACT

The present invention relates to a device including: a) means for measuring the reflectance or absorbance of light radiation by an area of the body or an extracorporeal element of an animal within a population of individuals; b) means suitable for receiving light-radiation reflectance or absorbance spectral data measured for each animal in the population of individuals; and c) data processing means suitable for determining, according to the received spectral data, a homeostatic emotional state for each animal, the animals being either in an unbalanced homeostatic emotional state or in a balanced homeostatic emotional state.

The present invention relates to a technology for non-invasive evaluation and prediction of the level of well-being, or more globally the emotional balance of an animal, or more generally of an individual or a population of individuals.

The present invention also relates to a device for measuring the reflectance or absorbance of a light radiation, by an area of the body or an extracorporeal element of an animal of a population of individuals, making it possible to determine, on the basis of measured spectral data, the homeostatic emotional state of each animal of the population.

Over the last 50 years, the question of the well-being, more globally of the emotional state, of animals living dependent on humans (livestock, pets, zoo animals, experimental animals, etc.) has become central in our societies.

More particularly, the question of animal emotions has been at the heart of many debates over the last 40 years. The capacity of animals to feel emotions is now widely accepted both in the scientific community and by decision-makers.

In France, since February 2015, animals are considered as “sentiment living beings”. These legislative changes have been supported by the considerable advance in ethological research (the study of animal behaviour) in the field of animal emotions.

In humans, the subjective component of an emotion can be measured by self-evaluation. In animals, this subjective dimension, i.e. how the individual “feels”, can only be estimated indirectly, by measuring the physiological and behavioural changes induced by the emotions. However, these changes are very useful, but are often more suitable for detecting the intensity of an emotion (strong or weak) than its quality (positive or negative emotion).

Techniques using the quantification of physiological markers can be quicker but more expensive for analyses (one marker per analysis and per animal), especially since it is sometimes necessary to repeat the analysis (in duplicate or triplicate) in order to avoid aberrant values and to obtain accurate measurement. Furthermore, this sometimes requires severe restraint of the animal and the introduction of a needle (syringe for sampling) when the measurement is carried out on the blood or plasma, which will contribute to inducing stress and therefore a bias in the measurement of the desired property. In a reverse approach, for laboratory animals, the emotional state, prior to the measurement of a biological marker in the context of clinical experimentation, will introduce a bias in the results obtained.

Negative emotions linked to fear or to pain were the first to be studied, since it is easier to experimentally induce a negative emotion than a positive emotion. The results of such studies make it possible to better understand the situations inducing negative emotions and to identify the useful indicators for animal well-being.

However, beyond the two “base” emotions of fear and pain, many other positive or negative emotional states exist, including in animals, in particular surprise, joy, boredom, sadness, vigilance, serenity, anger, etc.

Emotion can be defined as a positive or negative experience on the psychophysiological plane. This complex and intense response of an individual's state of mind reflects a reaction to biochemical (internal) and/or environmental (external) influences, now designated by the terms exposome and eco-exposome (totality of exposures (non-genetic) encountered over a lifetime). The exposome groups together various positive or negative factors, which are linked to the chemical components present in food, medical treatments (antibiotics, etc.), stress and anxiety. The eco-exposome groups together factors linked to environmental components such as climate change (heat), water and air pollution, or also the availability of food (plants, water, meat, etc.).

Hence, the emotional balance can be defined by a complete state of physical, psychological and social well-being (according to the WHO).

With regards to production animals, just as for laboratory animals, pets and zoo animals, today consideration of animal well-being or emotional state is essential not only among citizens, whether consumers or otherwise, and among farmers, but also among all the actors involved in the growth and trade of animals, products of animal origin or animal experimentation.

The concept of animal well-being animal has existed in European (EU) law since 1992. First established symbolically, today it is a constraining standard (europarl.europa.eu/RegData/etudes/STUD/2017/583114/IPOL_STU(2017)583114_EN.pdf).

In this context, the Welfare Quality® project initiated and financed by the European Union currently constitutes a reference framework from which protocols have been constructed and developed, in order to include a large number of production species (pigs, laying hens, chickens for fattening, cattle except calves, sheep, goats, horses and turkeys). Twelve criteria arising from four guiding principles have been defined (appropriate feed, appropriate shelter, good health, appropriate behaviour) which gives rise to multiple measurements performed in practice. These well-being criteria have been developed from “five fundamental liberties” (absence of hunger, thirst and malnutrition; absence of fear and distress; absence of physical and thermal stress; absence of pain, lesions and disease; the possibility for the animal to express the normal behaviours of its species) which include individual measurements performed on the animal and evaluation of resources.

Moreover, in the context of the study of sentience and the evaluation of the human emotional state, the study of different emotions and their development is of primary importance during behavioural studies, whether in the field of sociology, marketing, or even security. This being said, the methods for evaluating emotions of a human individual in a given situation often introduce a bias, because the individual knows he is being observed, evaluated or even manipulated; or are simply not possible without interfering directly with the individual.

In this context, the objective and non-invasive practical evaluation of the well-being, state or emotional balance of an animal or a human individual would appear to be a major and essential issue. Among other things, during this evaluation, it is a question of removing the anthropomorphic nature of the question, in other words that which is linked to our own human psychic experience, or overcoming the experimental bias linked to the fact that the animal or the individual is being manipulated or knows that they are being observed.

Today, for practical use in the field, many well-being or emotional balance assessment grids have been developed with varying degrees of complexity that are more or less suitable to all animal species.

More specifically, there is currently a lack of a simple specific tool, that is inexpensive, non-invasive and non-time-consuming in its implementation, and is capable of globally and objectively taking into account the biology of the species, the different stages of development and the environmental conditions.

The object of the present invention is to develop a non-invasive, objective, simple, quick and precise approach to the situation of well-being, the level of stress or more globally the emotional balance of the animal, which is also designated in the present application by the term “emotional homeostasis”.

The measurement approach employed here uses reflectance or scattering of light, in particular spectroscopy in the near or mid-infrared.

The near-infrared is characterised by a wavelength of approximately 700 to 2000 nm, and the mid-infrared by a wavelength of approximately 2000 nm to 20 μm.

Near-infrared and mid-infrared spectroscopy has been widely used in industry for a number of years. It includes a wide range of techniques, the most common being absorption spectroscopy. This technique makes possible a non-invasive analysis on the basis of varied biological samples (skin, urine, faeces, etc.), without using a solvent or preparation, in order to determine the composition of a sample, its humidity level and its protein content.

Infrared spectroscopy exploits the fact that molecules have specific frequencies at which they rotate or vibrate, corresponding to discrete energy levels (vibratory modes). The infrared spectrum of a sample is established by passing a beam of infrared light through this sample. The examination of the transmitted light indicates the quantity of energy absorbed at each wavelength.

Thus, a direct global molecular signature (equivalent to a fingerprint), synthesising in a single piece of information the presence of a set of chemical molecules characteristic of stress, a state of anxiety or unhappiness (cortisol, corticosterone, vasopressin, etc.), but also characteristic of well-being (γ-aminobutyric acid (GABA), ocytocin . . . ), can be obtained by analysis of the infrared spectrum of a liquid sample (such as a blood or tear sample, for example) or of a solid sample (skin, mucosa).

This “global signature” is defined here as being the physiological and/or physiopathological reflex encompassing all of the biological and hormonal mediators released in an individual in response to a situation.

The obtaining of such data makes it possible to characterise the state of well-being or the emotional balance or the emotional homeostasis of an animal, including a human individual.

Thus, the present invention relates to a method for determining the homeostatic emotional state of an animal using a device according to the invention, wherein said device measures a difference in reflectance or absorbance of light of an area of the body or of an extracorporeal element of said animal, which makes it possible to distinguish between two homeostasis emotional states, a state of balance and a state of imbalance.

The invention also relates to a method for determining the homeostatic emotional state within a population of animals, potentially comprising at least one animal for which the emotional homeostasis is in a state of imbalance and at least one animal for which the emotional homeostasis is in a state of balance, comprising subjecting said population to a device which separates them into:
i. a sub-population of animals in a state of emotional imbalance;
ii. a sub-population of animals in a state of emotional balance;
   wherein the device identifies the animals in a state of emotional imbalance on the basis of the measurement of a difference in reflectance or absorbance of light of an area of the body or of an extracorporeal element between animals of the population.

The present invention also relates to a device comprising:
a) means for measuring the reflectance or absorbance of a light radiation by an area of the body or an extracorporeal element of an animal within a population of individuals,
b) means suitable for receiving spectral data of reflectance or absorbance of a light radiation measured for each animal of said population of individuals, and
c) data processing means suitable for determining, on the basis of the spectral data received, a homeostatic emotional state of each animal, said animals being either in an imbalanced homeostatic emotional state, or in a balanced homeostatic emotional state.

The present invention also relates to the use of this device for determining the homeostatic emotional state of an animal.

The present invention also concerns the use of this device for determining a homeostatic emotional index of a population of animals, said use comprising:
a. subjecting said population to a device, enabling the measurement of the reflectance or absorbance of a light radiation by an area of the body or an extracorporeal element of each animal of the population,
b. calculating an individual homeostatic emotional index for each animal of the population, based on the measured reflectance or absorbance,
c. calculating a homeostatic emotional index of the population corresponding to the average of the individual indices calculated in step b.

### DETAILED DESCRIPTION OF THE INVENTION

The present invention relates to a device comprising:
a) means for measuring the reflectance or absorbance of a light radiation by an area of the body or an extracorporeal element of an animal within a population of individuals,
b) means suitable for receiving light-radiation reflectance or absorbance spectral data measured for each animal in the population of individuals; and
c) data processing means suitable for determining, on the basis of the received spectral data, a homeostatic emotional state for each animal, said animals being either in an unbalanced homeostatic emotional state or in a balanced homeostatic emotional state.

The expression “means suitable for” is synonymous with “means configured for”.

In the context of the present invention, “emotional homeostasis” is defined as the capacity of an animal to maintain or regain its psychological, emotional and somatic functioning balance despite external constraints.

The emotional homeostasis can be in a balanced state, which indicates a state of well-being, or in an unbalanced state, which mainly indicates or predicts negative emotions such as a state of unease, sadness, anger or stress for example, but also positive emotions such as excitement, sudden joy or surprise.

In the case of the present invention, an imbalance of the emotional homeostasis is not only an imbalance due to negative emotions but can encompass positive emotions. More specifically, the methods according to the invention can serve to identify the expression of positive emotions even in the absence of a facial expression; indeed an animal, human or non-human can sense joy or surprise without expressing this through characteristic facial expressions.

This concept of emotional homeostasis is linked to the concept of homeostasis in the physiological sense, which corresponds to the capacity of a system to maintain the balance of its internal environment, whatever the external constraints. On the scale of an organism, it involves a set of parameters which must remain constant or adapt to specific needs, such as, for example, body temperature, glycaemia, blood pressure or heartbeat. This physiological homeostasis is often controlled by hormones.

The term “homeostasis”, well established in chemistry and physiology, has thus been extended to physiological and sociological phenomena. More specifically, animals, face daily attacks of diverse nature, losses, frustrations, fears or threats, evoking negative and feared consequences; but also emotions such as joy, surprise or serenity, heralding positive and/or hoped-for consequences.

The sentiments and emotions linked to these various external factors can call into question the individual emotional homeostasis, which can therefore again be defined as the psychological, emotional and somatic balance.

This concept of emotional homeostasis includes all forms of stress, be they nutritional, environmental or linked to livestock farming practices.

The emotional homeostasis is linked to the exposome, which groups together all the exposures to environmental factors to which an animal is subjected from its conception. It is also linked to the concept of animal eco-exposome, which is defined as being the total internal exposure to anthropogenic and natural chemical substances, to their biotransformation products, and to endogenous signalling molecules which can be sensitive to an anthropogenic chemical exposure during the life of a living organism.

Within the meaning of the present invention, the terms “light” and “light radiation” are interchangeable, and are used interchangeably in the present description; they designate both the stimulus of an area of the body or an extracorporeal element of an animal, consisting of the exposure of said area or said element to a light radiation defined by a specific wavelength.

Within the meaning of the present invention, the observed “difference” corresponds to a detectable difference observed between the raw measurement obtained by the measurement device, and a reference measurement; or to a difference observed between two measurements carried out on two distinct animals.

Near-infrared spectroscopy (or NIRS) is a technique for measurement and analysis of reflection spectra in the long wavelength range extending from 700 to 2000 nm (the near-infrared).

According to an embodiment of the invention, the spectral data obtained correspond to reflectance or absorbance data of a light radiation in the near-infrared, for which the wavelength is in the range from 700 to 2000 nm, and more specifically from 908 to 1676 nm.

The term “reflectance”, also referred to as reflection factor, designates the proportion of light reflected by a surface.

The term “absorbance”, also referred to as optical density, designates the capacity of a medium to absorb the light which passes through it.

The measurement of the reflectance or absorbance of a light radiation by an area of the body or an extracorporeal element of an animal, enables “spectral data” to be obtained, which are then differentiated, in other words compared with other data, i.e. reference measurements, or spectral data measured on one or more other animals.

The term “reference measurement” also referred to as “reference value” designates values determined in animals of the same species as the tested animal species, previously or simultaneously with the implementation of the method, said animals being in a “basal state” for determining the reference values corresponding to a state of emotional balance, or in a state of stress for determining the reference values corresponding to a state of emotional imbalance. The method according to the invention can be based on two reference values, each being representative of a state, or on one or more averages of a plurality of representative values of a state, or on one or more medians of a plurality of representative values of a state, or even on one or more ranges of representative values of a state. The reference values can be so-called “threshold” values which enable two sub-populations to be separated, into those for which the measurements are greater than the threshold value, and those for which the measurements are less than the threshold value. Such reference values can easily be determined by a person skilled in the art using his general knowledge.

Once the measurements are obtained, the device will recognise the animal as being in a balanced homeostatic emotional state or in an unbalanced homeostatic emotional state (stressed, for example) on the basis of measurements taken from the animal, and this compared to control standards (reference values) recognised by the apparatus.

Typically, this operation will be carried out via a computerised comparison of data received from the animal with preprogrammed standards (reference values), such that the animal can be identified as being in emotional imbalance or in emotional balance.

The invention is characterised in that the device comprises data processing means suitable for differentiating the reflectance or absorbance data measured on each animal, in order to evaluate the homeostatic emotional state of the animal. These data processing means can, in particular, be used to compare the data obtained with other spectral data, in particular with reference values previously stored in said processing means.

In an embodiment, the device according to the invention also comprises spectral data storage means.

Once analysed and identified as being in an unbalanced homeostatic emotional state or in a balanced homeostatic emotional state, the device will provide a means for separating the animals and orienting these in an area containing animals which have been subjected to the same analysis and have given the same results.

The invention also relates to a method for determining the homeostatic emotional state within a population of animals potentially comprising at least one animal for which the emotional homeostasis is in a state of imbalance and at least one animal for which the emotional homeostasis is in a state of balance, comprising subjecting said population to a device which separates them into
i. a sub-population of animals in a state of emotional imbalance;
ii. a sub-population of animals in a state of emotional balance;
   wherein the device identifies the animals in a state of emotional imbalance on the basis of the measurement of a difference in reflectance or absorbance of light of an area of the body or an extracorporeal element between animals of the population.

According to an embodiment, this method is characterised in that the device is coupled to data processing means suitable for differentiating the reflectance or absorbance data measured for an animal in a state of emotional imbalance from those of an animal in a state of emotional balance, which makes it possible to separate the population into two sub-populations of animals, one in a state of emotional imbalance and the other in a state of emotional balance.

According to an embodiment of the invention, the method and the device according to the invention are characterised in that the measurement of the reflectance or absorbance of light is performed on an area of the body chosen from the skin and the superficial mucus membranes of an animal. This measurement can also be performed on at least two body areas of the same animal.

More particularly, the measurement of reflectance is carried out on the skin, in particular on the skin of the ear, the skin of the axilla, and the skin of the udder of a cow.

More particularly, the measurement of reflectance is carried out on a mucosa, which can in particular be the mucous membrane of the vulva.

Within the meaning of the invention, the area of the body on which the light radiation is applied is naked and not tattooed.

According to an embodiment of the invention, the measurement is a measurement of reflectance or absorbance, which is performed on an extracorporeal element which is a bodily fluid, a digestion residue, a secretion or a bodily exudate, and can be chosen from the group comprising urine, tears, faeces and sweat.

According to an embodiment of the invention, the measurement of reflectance or absorbance of light of an area of the body is performed by contact with said area of the body.

According to another embodiment of the invention, the measurement of reflectance or absorbance of light of an area of the body is performed by a remote measurement of said area of the body.

Here, the term “remote measurement” shall mean a measurement carried out at a distance of between 1 cm and 100 m, in particular between 1 cm and 70 m, or even between 1 cm and 60 m, between 1 cm and 50 m, between 1 cm and 40 m, between 1 cm and 30 m, between 1 cm and 20 m, between 1 cm and 15 m, between 1 cm and 10 m, between 1 cm and 5 m, for example. The limit being the spatial resolution enabling the area of the body studied to be targeted and distinguished.

The remote measurement can also be carried out using a portable or fixed device. Such a device can be a portable spectrometer, a multispectral or hyperspectral camera, for example.

According to another embodiment, the invention also relates to a method for determining a homeostatic emotional index of a population of animals comprising:
a) subjecting said population to a device, enabling the measurement of the reflectance or absorbance of light by an area of the body or an extracorporeal element of each animal of the population,
b) calculating an individual homeostatic emotional index for each animal of the population on the basis of the measured reflectance or absorbance of light,
c) calculating a homeostatic emotional index of the population corresponding to the average of the individual indices calculated in step b.

According to an implementation of the invention, the calculating of an individual homeostatic emotional index of an animal is performed via a computerised comparison of data received from the animal with reference values, using suitable data processing means, such that the animal can be identified as being in emotional imbalance or in emotional balance.

According to another implementation of the invention, the calculating of the homeostatic emotional index of the population is performed by comparison of the index calculated in step c) with a reference value.

The reference values for an individual animal or for a population of animals can easily be determined by a person skilled in the art.

According to an embodiment, the device comprises means for measuring the reflectance or absorbance of light of an area of the body or an extracorporeal element of each animal of the population enabling a measurement by contact with the animal.

Preferably, this involves a device comprising means for measuring the reflectance of an area of the body enabling a measurement by contact with the skin of an area of the body of the animal.

In a particular embodiment, the device enabling the measurement by contact with the animal is an individual device carried by each animal of the population.

According to another particular embodiment, the individual device carried by each animal records and/or transmits the measurement of the reflectance or absorbance of light by an area of the body or an extracorporeal element of each animal.

According to another particular embodiment, the individual device carried by each animal records and/or transmits the individual homeostatic emotional state of each animal.

This transmission is preferably carried out to a server enabling the storage of data, and particular to a set of servers in a network (“cloud” type infrastructure).

In this server or this set of servers, data collected on multiple animals, of various species and under various conditions, will be stored in such a way as to constitute a “database”, able to be used to supply reference values for the implementation of the methods of the invention.

In an advantageous embodiment, the device comprising means for measuring the reflectance of light of an area of the body of each animal of the population is a device enabling a remote measurement, which records and/or transmits: the measurement of the reflectance or the absorbance of light and/or the state and/or the individual homeostatic emotional index of each animal.

According to an embodiment, the animal is chosen from the group consisting of mammals, cold-blooded animals, and birds.

In particular, cold-blooded animals include reptiles, fish, insects or amphibians.

More particularly, the mammal is chosen from the group consisting of primates, canids, felids, ovines, caprines, porcines, equines, ungulates, suids, elephantids, cetaceans, lagomorphs and rodents.

According to a particular implementation, the mammal is a non-human mammal.

Within the meaning of the invention, the expression “population of animals” or “population of individuals” designates a set of animals of the same species (in other words able to reproduce among themselves), comprising at least 10, 20, 30, 40, 50, 100, 200, 300, 400, 500, 1000, 1500, 2000 or more individuals. This population of animals will be, in particular, raised on the same farm.

Finally, the invention also relates to the use of a device comprising means for measuring the reflectance or absorbance of light of an area of the body or an extracorporeal element of an animal, as well as means for receiving and processing data, in order to determine the level of emotional homeostasis of said animal.

The sorting of the population of animals according to a method according to the invention, will involve determining the reflectance and/or absorbance characteristics of each individual using light, in particular a near-infrared light radiation.

For example, for a measurement by reflectance, the animal can be illuminated by the light of a predefined spectrum, and the spectral characteristics of the reflective light are measured.

For detection by absorbance, the device is such that it measures the light which is absorbed by a sample of the extracorporeal element, and thus measures the spectral characteristics of the light which is absorbed by the sample.

According to the present invention, said device uses data processing means suitable for differentiating the spectral data, in particular in the near-infrared, obtained from animals in an unbalanced homeostatic emotional state and from animals in a balanced homeostatic emotional state; then enables the animals to be separated and distinguished on the basis of said spectral data.

Thus, the present invention also relates to a device for measuring the reflectance or absorbance of light in order to implement a method according to the invention, comprising processing means suitable for receiving reflectance or absorbance data measured for each animal (either on an area of the body, or on an extracorporeal element of said animal), and enabling the homeostatic emotional state of the animal to be determined on the basis of these data.

Before implementing the methods of the present invention, control animals can be tested in order to verify the spectral data, the reflectance and/or absorbance of a selection of animals in an unbalanced homeostatic emotional state and of animals in a balanced homeostatic emotional state, and this in order to determine reference values. More specifically, it is very well known to unbalance the emotional state of an animal by application or external factors creating and inducing a stress, and to do so in a directed and controlled manner.

Once these reference values have been defined, they can be used to generate an algorithm which will be used to discriminate the homeostatic emotional state of the tested animals.

Such an algorithm can then be used in the device which, as described above, contains the means for testing each of the animals in a general population, then, on the basis of results compared with reference values, to select animals in an unbalanced homeostatic emotional state or balanced homeostatic emotional state, and to separate them as a consequence.

A person skilled in the art is capable of generating an appropriate algorithm for a use of said device on the basis of an identification of spectral data of the animals to be distinguished.

According to an embodiment, the device according to the invention comprises data processing means suitable for carrying out the following successive steps:
a) principal component analysis (PCA) of a first matrix comprising N rows and P columns, each row corresponding to the spectral data of an animal n=1 to N, for each wavelength p=1 to P of the spectral data, so as to reduce the number of columns and thus obtain a second matrix comprising P′≤P columns;
b) cleaning aberrant spectra and denoising spectra using a mathematical preprocessing, in order to obtain a third matrix comprising N′≤N rows; and
c) linear discriminant analysis (LDA) of the third matrix obtained in step (b) in order to determine the state of emotional homeostasis of an animal on the basis of its spectrum.

According to an implementation of the invention, said data processing means are moreover suitable for carrying out the following action:
d) optimising and validating the model.
   FIG. 1 schematically shows the various data processing steps.

The set of the spectral data obtained constitutes N spectra (also designated “spectral data”), each spectrum corresponding to one animal, numbered n=1 to n=N. These N spectra are concatenated into a first matrix containing N rows and P columns, corresponding to the P wavelengths of the spectrum.

The first matrix obtained thus contains N rows and P columns.

The numbers N,N′, P and P′ are integers.

A data reduction operation is performed by principal component analysis (PCA). The matrix then has fewer columns (for as many rows), which enables faster processing for an identical computing power.

The second matrix comprises P′SP columns.

A cleaning operation of aberrant near-infrared spectra is then performed on the basis of the PCA results (T2 Hotelling values and/or F-residuals). The matrix thus cleaned comprises a few less rows, i.e. N′≤N rows.

A mathematical preprocessing of denoising and normalisation is then carried out on all the remaining spectra (first derivative of FIG. 1), in order to obtain a third matrix comprising N′≤N rows, comprising the spectra ready to be used in modelling.

The modelling algorithm consists of a linear discriminant analysis (LDA), and reveals whether the spectrum which has been collected comes from an individual with a balanced or unbalanced homeostatic emotional state.

According to a particular implementation, a last step (d) is performed in order to optimise and validate the model.

Two steps are employed for optimising and validating the model (cross validation and external validation) and the performances obtain are represented in a table (confusion matrix).

Once optimised, the model obtained based on the training database can be used to classify new near-infrared spectra.

The result of this classification and the calculated index makes it possible to determine whether the spectral data collected from an individual n correspond to an individual presenting a balanced or unbalanced emotional homeostasis.

The present invention also relates to the use of the device according to the invention for determining a homeostatic emotional index of a population of animals comprising:
a) subjecting said population of animals to said device, enabling the measurement of the reflectance or absorbance of a light radiation by an area of the body or an extracorporeal element of each animal of the population,
b) calculating an individual homeostatic emotional index for each animal of the population, based on the measured reflectance or absorbance,
c) calculating a homeostatic emotional index of the population corresponding to the average of the individual indices calculated in step b.

According to an embodiment, the determining of the emotional homeostasis index of the population is performed by comparison of the index calculated in step c) with a reference value.

### FIGURES

FIG. 1. Schematic representation of the processing of the measured data. The following abbreviations are used: BDD: Binary Decision Diagram; PCA: principal component analysis; N×P matrix: has N rows and P columns.

FIG. 2. Experimental protocol for tests on chicks.

FIG. 3. Emotional homeostasis index of the chicks, before and after stress.

FIG. 4. Level of plasma FITc-dextran (in μM) of control animals (control) and animals after stress.

FIG. 5. Emotional homeostasis index of calves, before and after stress.

FIG. 6. Experimental protocol for tests on rats.

FIG. 7. Emotional homeostasis index of the rats, before and after stress.

FIG. 8. Level of ACTH (pg/mL) before and after stress, according to two representations (A) and (B).

FIG. 9. Emotional homeostasis index of the chicks, calves and rats, before and after stress.

FIG. 10. Correction of the homeostatic emotional index of the chicks, calves and rats after subtraction of the basal data.

### EXAMPLES

The examples presented below are purely illustrative and do not limit the claimed invention in any way.

### Material and Methods:

### Material MicroNIR Onsite: Spectrometer and Measurement Protocol

A MicroNIR Onsite (Viavi, Santa Rosa, CA, USA) near-infrared spectrometer with remote probe (908-1676 nm, with a step of 6 nm and a passband FWHM <1.25% of the central wavelength) was used. The light source consists of 2 tungsten lamps incorporated under vacuum. After taking the temperature, a measurement of the instrumental noise and the white reference was performed at regular intervals. The white reference is made on an external Spectralon. The reference measurements are made every 10 minutes; time adjusted on the basis of the environmental conditions. These reference measurements can work in reflectance using the MicroNIR™ Pro v2.5 software (Viavi, Santa Rosa, CA, USA).

### Measurement Method:

The measurement is performed on contact, by positioning the spectrometer perpendicular to the measurement area. Each measurement corresponds to the average of 100 spectra, which each integrates the collection over 1 ms. An observation corresponds to a plurality of averaged measurements. The number of measurements per observation varies on the basis of the experimental conditions. For each experiment, measurements are performed in the basal condition i.e. balanced homeostatic emotional condition, and after stress i.e. unbalanced homeostatic emotional condition.

### Data Processing: Chemometrics

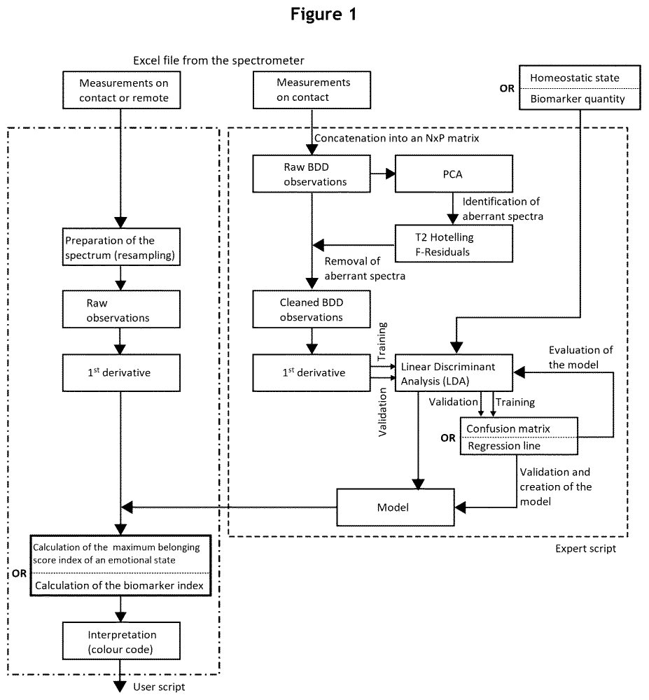

All of the measurements collected are processed according to the scheme shown in FIG. 1. After cleaning the spectral data base consisting of visualising aberrant spectra using The Unscrambler@ Multivariate Data Analysis (v. X; CAMO A/S, Oslo, Norway) then performing a principal component analysis (PCA) associated with an influence graphic representing the T² Hotelling versus F-residuals (The Unscrambler® X) (Agelet and Hurburgh 2010), the aberrant spectra are discarded in order to continue the data analysis. The remaining spectra from repetitions are next averaged and then undergo a mathematical preprocessing for baseline correction, smoothing and derivative, diffusion correction (SNV and MSC), etc. (Bertrand and Dufour 2005, Agelet and Hurburgh 2010) in order to remove or reduce variations in intensity linked to undesirable factors and to increase the signal of the “useful” chemical information. These preprocessing tasks are optimised on the basis of the animal species and the location of the measurement.

### Example 1: Stressed and Unstressed Chicks

### Material and Methods:

### Animals

Chicks from the strain ROSS PM3 (“Socavic” hatchery at Monferran-Savès (32, Gers)) were obtained on the day of hatching (d0). On arrival, the chicks were weighed then allocated to “enriched” cages in order to meet animal well-being standards. The parameters of temperature, humidity and lighting of the livestock building were determined according to the Aviagen® (Aviagen, 2018) guide. The animals received feed and water ad libitum. The feed corresponded to a conventional formulation reflecting the usual livestock conditions and responding to the nutritional needs of the animals. In brief, the feed was composed of a grain corn base (between 38 and 40% depending on age) supplemented by wheat (qs 20%) for a total intake of 60% cereals and supplemented by a source of protein (soybean meal, 48% total nitrogenous material) representing 30% of the formula and soybean oil making up 4% of the feed.

### Stress Procedure:

According to the protocols described in (Wall et Cockrem, 2010) et (Post, Rebel, et Huurne, 2003), corticosterone (20 mg/l) was added in the drinking water in order to mimic a natural response to stress in the animal. The animals received this preparation for 3 consecutive days, from d11 to d13. Following this period, the chickens again received water ad libitum, without corticosterone.

### Measurement of Intestinal Permeability:

A stress session provoked in the neonatal period (d11 to d13) leaves an imprint in adulthood resulting in a micro-inflammation of the intestinal mucous membrane and an increase in intestine paracellular permeability. This post-stress state results in an increase in the level of FITC-Dextran in the bloodstream of stressed animals compared with control animals (Baxter et al., 2017). Here, the intestinal paracellular permeability was evaluated using a biomarker: 4 kDa FITC-Dextran (fluorescein isothiocyanate dextran) coupled with fluorescein. At D35, the animals received (1 mL/animal) a solution of 8.32 mg/kg FITC-Dextran by gavage (diluted in NaCl) (Baxter et al., 2017; Maguey-Gonzalez et al., 2018; Vicuna et al., 2015). An hour after gavage, a blood sample was taken from the wing of the animal and then the level of FITC-Dextran was measured in the plasma.

### Experimental Protocol According to the Invention;

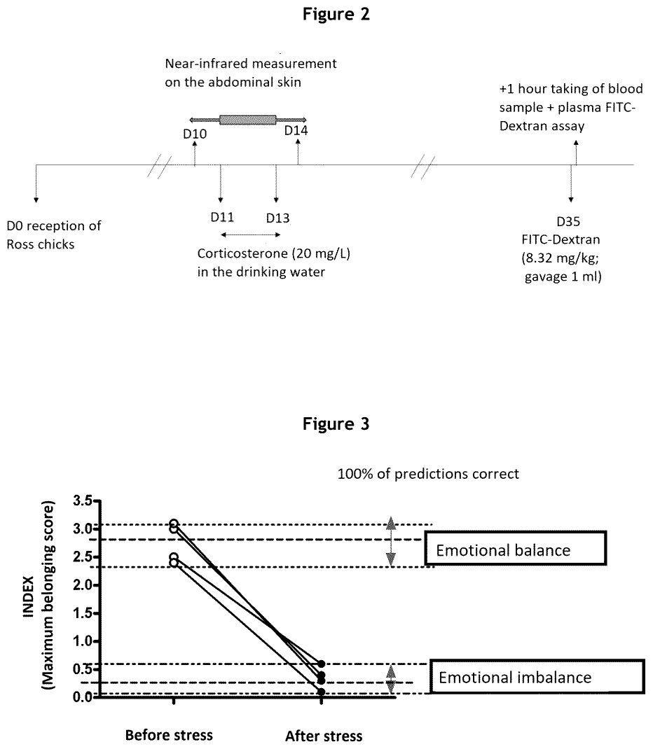

FIG. 2 illustrates the following experimental protocol.

10 days after reception of the animals, near-infrared spectra were measured under the wing of the chicks so as to define a basal state (emotional balance).

From the 11th day and until the 13th day, corticosterone was taken by adding it in the drinking water. A new series of measurements is performed on the 14th day after defining the state of stress and thus defining a state of emotional imbalance.

On d35 in the adult chicken, FITC-Dextran was administered by gavage. 1 hour after gavage, a blood sample was taken from the wing vein of the animal, in order to measure the level of plasma FITC-Dextran, markers of the long-term imprint (adult age) of the emotional imbalance caused by the addition of corticosterone in the drinking water of the chick.

### Results:

TABLE 1
Principal Component Analysis. The results are presented
in the following table (underwing measurement).

| Chick | Measurement | Measurement | State of emotional | Prediction |
|---|---|---|---|---|
| number | before stress | after stress | homeostasis | value |
| 1 | 2.46 | 0.62 | Balance before | Correct in |
| 2 | 3.02 | 0.35 | stress, imbalance | 100% of |
| 3 | 3.08 | 0.31 | after stress | cases |
| 4 | 2.44 | 0.13 | | |

FIG. 3 shows the distinct distribution of the emotional state in the chicks: “before stress” and “after stress” on the basis of a score designated the “maximum belonging score” defined on the basis of cutaneous measurements performed in the near-infrared spectrum.

In this experiment, 100% of the animals were correctly predicted: emotional balance vs. emotional imbalance (n=4; (p=0.0012 t test); the medians are shown here).

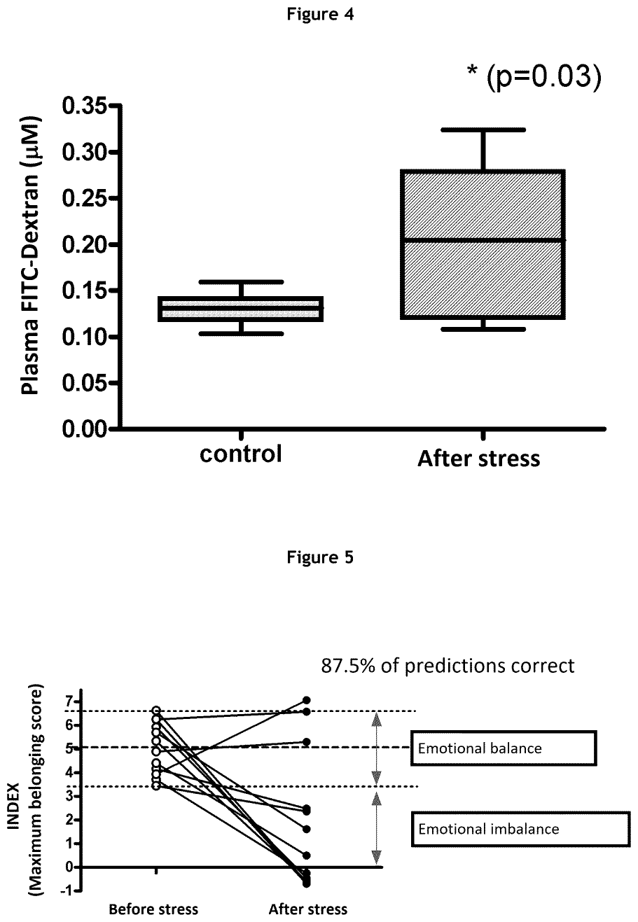

FIG. 4 clearly shows the incidence of an emotional imbalance occurring during the neonatal period on the intestine paracellular permeability at adult age. A neonatal stress causes a significant increase (n=12, p=0.03 Mann Whitney test; average±SEM) at adult age of the plasma FITC-Dextran levels vs. control, reflecting an alteration in the intestinal barrier induced by an early administration of corticosterone in the drinking water of the young animal.

### Example 2: Stressed and Unstressed Calves

### Material, Methods and Experimental Protocol:

Twelve Prim'Holstein calves when used in these studies. Fifteen days after their birth, after application of a local analgesic, the animals were subjected to dehorning after having been placed in a containment cage intended for this purpose.

As previously described, near-infrared reflection measurements were performed on the skin inside the ear before and after dehorning, so as to respectively define a basal state (emotional balance) and a stressed state (emotional imbalance).

TABLE 2
Principal Component Analysis. The results are presented
in the following table (measurement on the ear)

| | Measure- | Measure- | | |
|---|---|---|---|---|
| | ment | ment | | |
| Calf | before | after | State of emotional | Prediction |
| number | stress | stress | homeostasis | value |
| 1 | 3.71 | −0.25 | Balance before stress, | correct |
| 2 | 4.16 | 2.48 | imbalance after stress | correct |
| 3 | 3.44 | 2.36 | | correct |
| 4 | 5.92 | −0.46 | | correct |
| 5 | 6.25 | 2.87 | “Balance” before and | poor |
| | | | after stress | |
| 6 | 3.93 | 3.44 | “Balance” before and | poor |
| | | | after stress | |
| 7 | 6.26 | −0.70 | Balance before stress, | correct |
| | | | imbalance after stress | |
| 8 | 4.89 | 3.16 | “Balance” before and | poor |
| | | | after stress | |
| 9 | 5.70 | 1.61 | Balance before stress, | correct |
| 10 | 6.63 | −0.65 | imbalance after stress | correct |
| 11 | 5.34 | −0.58 | | correct |
| 12 | 4.40 | 0.50 | | correct |

In this example, scores higher than a threshold value are obtained before stress, and are considered to be associated with an emotional balance.

In the present case, there are three animals (calves 5, 6 and 8) which, after dehorning, have not been detected as being in a state of emotional imbalance by applying the method of the invention. However, the method has nevertheless made it possible to obtain 87.5% correct predictions.

FIG. 5 also illustrates the distinct distribution of the emotional state in the calves: “before dehorning” and “after dehorning” on the basis of a score defining the maximum belonging score on the basis of skin measurements performed in the near-infrared spectrum.

Here 87.5% of the animals were correctly predicted: emotional balance vs. emotional imbalance (n=12; (p=0.008 t test); the median is shown here).

### Example 3: Stressed and Unstressed Rats

### Material and Methods:

### Animals:

Wistar rats (200-250 g) (Janvier SA, The Genest St Isle, France), weighing from 220 to 250 g, housed in polypropylene cages in a temperature-controlled room (21° C.) and receiving a standard feed (UAR, Villemoisson, Epinay sur Orge) and water ad libtum, were used.

### Chronic Water Avoidance Stress (WAS):

All the WAS sessions were carried out at the same time of day (08:00 and 10:00) in order to limit the influence of circadian rhythms. The rats are placed on a narrow platform of (14 height×6×6 cm), itself placed at the centre of a basin of (55 cm length×37 cm width×26 cm height) filled with water up to 1 cm below the level of the platform. The stress session consists of placing the animal on the platform for 1 hour per day, over a period of 9 consecutive days (5+4 days interruption during the week-end).

### ACTH Measurement:

Blood samples were taken from the facial (submandibular) vein, before the first stress session and after the last stress session (30 minutes after). After centrifugation, assays of ACTH (adrenocorticotrophin) were performed using an EIA kit (Clinisciences ref LS-F5355-1, Nanterre, France)

### Experimental Protocol:

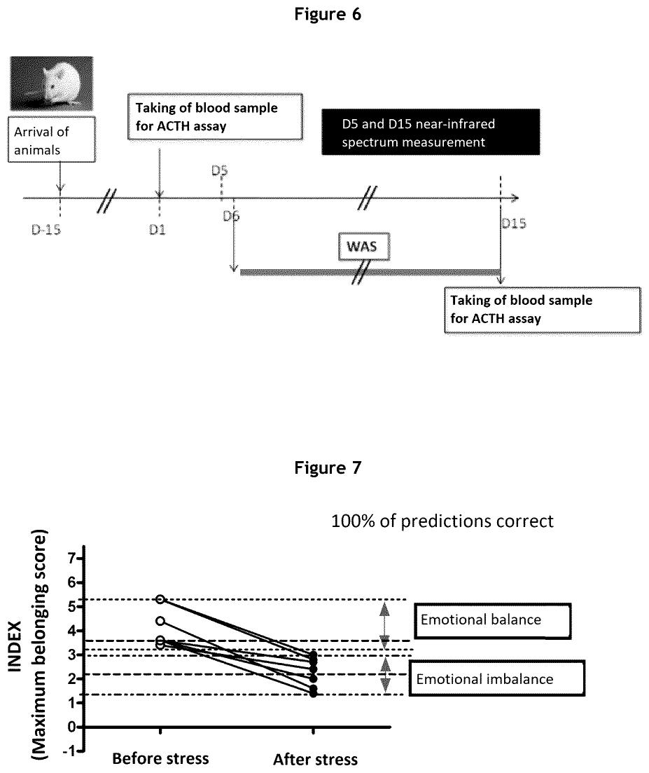

15 days after reception of the animals, a blood sample was taken in order to define the level of plasma ACTH, in the basal condition (emotional balance). Six days after this sampling, the rats are subjected daily to a chronic water avoidance stress (WAS) for 9 days (D6 to D15). One day after the first stress session (emotional balance) and 20 minutes after the last stress session (emotional imbalance), near-infrared spectra were collected at the ear, following the previously described protocol. On the last day of stress, a blood sample is taken in order to measure the level of plasma ACTH in a condition of emotional imbalance (cf. FIG. 6)

### Results:

TABLE 3
principal component analysis is performed
and the results are presented

| | Measure- | Measure- | | |
|---|---|---|---|---|
| | ment | ment | | |
| Rat | before | after | State of emotional | Prediction |
| number | stress | stress | homeostasis | value |
| 1 | 3.56 | 1.37 | Balance before | Correct in |
| 2 | 3.56 | 1.99 | stress, imbalance | 100% of |
| 3 | 4.42 | 1.59 | after stress | cases |
| 4 | 5.28 | 3.01 | | |
| 5 | 3.39 | 2.37 | | |
| 6 | 5.28 | 2.80 | | |
| 7 | 3.58 | 2.69 | | |

FIG. 7 shows the distinct distribution of the emotional state in the rats: before and after WAS on the basis of a score defining the maximum belonging score on the basis of skin measurements performed by near-infrared spectroscopy.

Here, 100% of the animals were predicted correctly: emotional balance vs emotional imbalance (n=7; p=0.0003 Wilcoxon test; the medians are shown here).

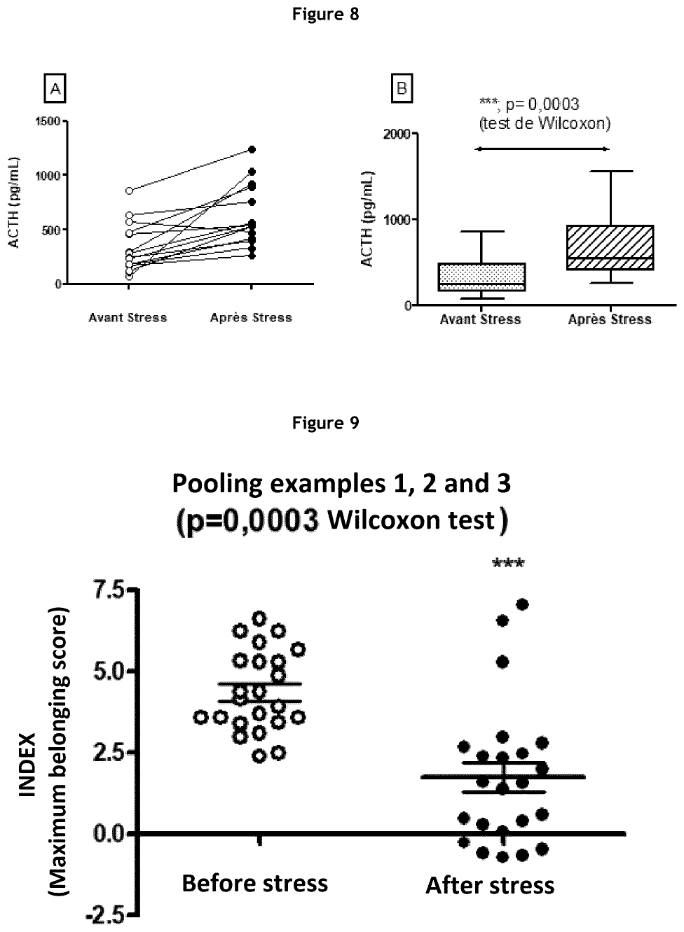

FIGS. 8A and 8B show the significant development of the levels of plasma ACTH, after a chronic stress in the rat (n=14).

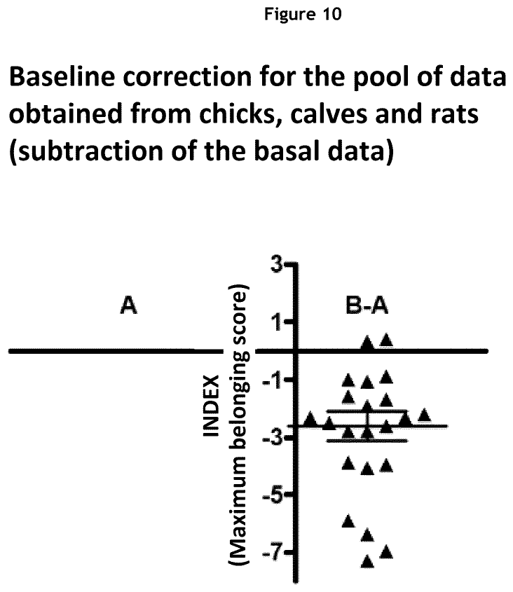

FIGS. 9 and 10 showed the significant robustness of the score defined for all species combined.

FIG. 9 corresponds to the representation of all the scores measured in the examples 1 (chicks); 2 (calves); 3 (rats).

### CONCLUSION

The examples presented here for three different animal species (bird, cattle, rodent), taken separately or together, show that the chemometric score from spectroscopic measurements performed with a light of near-infrared or mid-infrared wavelength by contact, according to the method of the invention, can predict the state of emotional balance or imbalance of an animal, with a reliability of 90-100%.

### BIBLIOGRAPHICAL REFERENCES

Bertrand and Dufour, La spectroscopie infrarouge et ses applications analytiques, 2nde édition, Collection Sciences et Technologiques Agroalimentaires, Tec & Doc Lavoisier, Paris (2006).
Lidia Esteve Agelet & Charles R. Hurburgh Jr. (2010) A Tutorial on Near Infrared Spectroscopy and Its Calibration, Critical Reviews in Analytical Chemistry, 40:4, 246-260, DOI: 10.1080/10408347.2010.515468
Wall J P, Cockrem J F. Effects of corticosterone treatment in laying Japanese quail. Br Poult Sci. 2010 April; 51 (2): 278-88. doi: 10.1080/00071661003745828. PMID: 20461589.
Post J, Rebel J M, ter Huurne A A. Physiological effects of elevated plasma corticosterone concentrations in broiler chickens. An alternative means by which to assess the physiological effects of stress. Poult Sci. 2003 August; 82 (8): 1313-8. doi: 10.1093/ps/82.8.1313. PMID: 12943303.
Baxter M F A, Merino-Guzman R, Latorre J D, Mahaffey B D, Yang Y, Teague K D, Graham L E, Wolfenden A D, Hernandez-Velasco X, Bielke L R, Hargis B M, Tellez G. Optimizing Fluorescein Isothiocyanate Dextran Measurement As a Biomarker in a 24-h Feed Restriction Model to Induce Gut Permeability in Broiler Chickens. Front Vet Sci. 2017 Apr. 19; 4:56. doi: 10.3389/fvets.2017.00056. PMID: 28470003; PMCID: PMC5396023.
Maguey-Gonzalez J A, Michel M A, Baxter M F A, Tellez G Jr, Moore P A Jr, Solis-Cruz B, Hernandez-Patlan D, Merino-Guzman R, Hernandez-Velasco X, Latorre J D, Hargis B M, Gomez-Rosales S, Tellez-Isaias G. Effect of humic acids on intestinal viscosity, leaky gut and ammonia excretion in a 24 hr feed restriction model to induce intestinal permeability in broiler chickens. Anim Sci J. 2018 July; 89 (7): 1002-1010. doi: 10.1111/asj.13011. Epub 2018 Apr. 30. PMID: 29708627.
Vicuña E A, Kuttappan V A, Tellez G, Hernandez-Velasco X, Seeber-Galarza R, Latorre J D, Faulkner O B, Wolfenden A D, Hargis B M, Bielke L R. Dose titration of FITC-D for optimal measurement of enteric inflammation in broiler chicks. Poult Sci. 2015 June; 94 (6): 1353-9. doi: 10.3382/ps/pev111. Epub 2015 Apr. 15. PMID: 25877413.

1. A device comprising:
a) means for measuring the reflectance or absorbance of light radiation by an area of the body or an extracorporeal element of an animal within a population of individuals;
b) means suitable for receiving light-radiation reflectance or absorbance spectral data measured for each animal in the population of individuals; and
c) data processing means suitable for determining, on the basis of the received spectral data, a homeostatic emotional state for each animal,
said animals being either in an unbalanced homeostatic emotional state or in a balanced homeostatic emotional state.

2. The device according to claim 1, wherein the spectral data correspond to near infrared light-radiation reflectance or absorbance data.

3. The device according to claim 1, wherein it is suitable for carrying out the measurement of reflectance or absorbance of a light radiation by the skin and/or the superficial mucosa of an animal.

4. The device according to claim 1, wherein it is suitable for carrying out the measurement of reflectance or absorbance of a light radiation by contact with the area of the body or with the extracorporeal element.

5. The device according to claim 1, wherein it is suitable for carrying out the measurement of reflectance or absorbance of a light radiation at a distance from the area of the body or from the extracorporeal element.

6. The device according to claim 1, wherein it is an individual device carried by each animal of the population.

7. The device according to claim 6, wherein said device records and/or transmits:
the measurement of the reflectance or absorbance of a light radiation by an area of the body or by an extracorporeal element of an animal, and/or
the homeostatic emotional state of an animal.

8. The device according to claim 1, wherein it is suitable for carrying out measurements on animals chosen from the group consisting of mammals, cold-blooded animals, and birds.

9. The device according to claim 8, wherein said animal is a mammal and is chosen from the group consisting of primates, canids, felids, ovines, equines ungulates, suids, elephantids, cetaceans, lagomorphs and rodents.

10. The device according to claim 1, wherein the data processing means are suitable for carrying out the following successive actions:
a) principal component analysis (PCA) of a first matrix comprising N rows and P columns, each row corresponding to the spectral data of an animal n=1 to N, for each wavelength p=1 to P of the spectral data, so as to reduce the number of columns and thus obtain a second matrix comprising P′≤P columns;
b) cleaning aberrant spectra and denoising spectra using a mathematical preprocessing, in order to obtain a third matrix comprising N′≤N rows; and
c) linear discriminant analysis (LDA) of the third matrix obtained in step (b) in order to determine the state of emotional homeostasis of an animal on the basis of its spectrum.

11. The device according to claim 10, wherein the data processing means are also suitable for carrying out the following additional action:
d) optimising and validating of a model.

12. A process for determining the homeostatic emotional state of an animal with the use of a device according to claim 1.

13. A process for determining a homeostatic emotional index of a population of animals comprising:
a. subjecting said population of animals to a device according to claim 1, enabling the measurement of the reflectance or absorbance of a light radiation by an area of the body or by an extracorporeal element of each animal of the population,
b. calculating an individual homeostatic emotional index for each animal of the population, based on the measured reflectance or absorbance obtained in step a,
c. calculating a homeostatic emotional index of the population corresponding to the average of the individual indices calculated in step b.

14. A process according to claim 13, wherein the determining of the homeostatic emotional index of the population is performed by comparing the index calculated in step c with a reference value.
